(19) United States
(12) Patent Application Publication
(10) Pub. No.: US 2017/0028095 A1
(43) Pub. Date: Feb. 2, 2017

(54) AIR PURIFIER FOR BRINGING GAS INTO CONTACT WITH PLASMA-TREATED LIQUID

(71) Applicant: Panasonic Intellectual Property Management Co., Ltd., Osaka (JP)

(72) Inventors: TATSUSHI OHYAMA, Osaka (JP); HIROKO MURAYAMA, Osaka (JP); MASAHIRO ISEKI, Osaka (JP)

(21) Appl. No.: 15/206,478

(22) Filed: Jul. 11, 2016

(30) Foreign Application Priority Data
Jul. 31, 2015 (JP) 2015-152785

(51) Int. Cl. A61L 9/22; A61L 9/16; B01D 50/00; B01D 47/16; B01D 46/00

(52) U.S. Cl. CPC A61L 9/22; B01D 47/16; B01D 46/0035; B01D 46/0038; B01D 50/006; A61L 9/16; B01D 2279/40; A61L 2209/14; A61L 2209/11

(57) ABSTRACT

An air purifier includes: a case having an air inlet and an air outlet; a tank that stores a liquid, the tank disposed in the case; a plasma generator that generates a plasma which is to be in contact with the liquid, the plasma generator including a pair of electrodes and a power supply which applies a voltage between the pair of electrodes; a fan that generates an air flow from the air inlet to the air outlet; a filter through which the air flow comes into contact with the liquid, the filter disposed in the case; and a controller that causes the power supply to start application of the voltage, and causes the fan to start generation of the air flow after elapse of a predetermined time period since the start of the application of the voltage.

### BACKGROUND

1. Technical Field

The present disclosure relates to an air purifier.

2. Description of the Related Art

In the related art, a known technology purifies water in a water tank of a humidifier unit of an air treatment device by using electric discharge.

For example, Japanese Patent No. 4656138 discloses an air treatment device including an electric discharge unit that produces active species by performing electric discharge, and an air purifier unit having a filter and a deodorizing member. In the air treatment device, air containing active species is supplied to the air purifier unit and the water in the water tank.

### SUMMARY

An air purifier according to an aspect of the present disclosure includes: a case having an air inlet and an air outlet; a tank that stores a liquid, the tank disposed in the case; a plasma generator that generates a plasma which is to be in contact with the liquid, the plasma generator including a pair of electrodes and a power supply which applies a voltage between the pair of electrodes; a fan that generates an air flow from the air inlet to the air outlet; a filter through which the air flow comes into contact with the liquid, the filter disposed in the case; and a controller that causes the power supply to start application of the voltage, and causes the fan to start generation of the air flow after elapse of a predetermined time period since the start of the application of the voltage.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1 is an illustration depicting an example of configuration of an air purifier according to a first embodiment;

FIG. 2 is a flow chart illustrating a first example of operation of the air purifier according to the first embodiment;

FIG. 3 is a flow chart illustrating a second example of operation of the air purifier according to the first embodiment;

FIG. 4 is a graph illustrating a variation in the concentration of hexanal with respect to an elapsed time of plasma treatment;

FIG. 5A is a flow chart illustrating a third example of operation of the air purifier according to the first embodiment;

FIG. 5B is a timing chart illustrating the timing of generation of a plasma and the timing of generation of an air flow in the operation illustrated in FIG. 5A;

FIG. 6A is a flow chart illustrating a fourth example of operation of the air purifier according to the first embodiment;

FIG. 6B is a timing chart illustrating the timing of generation of a plasma and the timing of generation of an air flow in the operation illustrated in FIG. 6A;

FIG. 7 is a flow chart illustrating a fifth example of operation of the air purifier according to the first embodiment;

FIG. 8 is a flow chart illustrating a sixth example of operation of the air purifier according to the first embodiment;

FIG. 9 is a graph illustrating a result of the concentration of odor substances, detected by using a VOC sensor, in the vicinity of an air outlet when a plasma is generated after an air flow is stopped in the example of operation illustrated in FIG. 7;

FIG. 10 is a graph illustrating a result of the concentration of odor substances, detected by using a VOC sensor, in the vicinity of an air outlet when a time period is provided in which a plasma and an air flow are generated concurrently in the example of operation illustrated in FIG. 7; and

FIG. 11 is a configuration diagram illustrating an example of an air purifier according to a second embodiment.

### DETAILED DESCRIPTION

### Overview of Embodiments

An air purifier according to an aspect of the present disclosure includes: a case having an air inlet and an air outlet; a fan that generates an air flow so that a gas is taken into the case through the air inlet or a gas is discharged to the outside of the case through the air outlet; a tank that is disposed in the case and that stores a liquid; a plasma generator that includes a pair of electrodes and a power supply which applies a voltage between the pair of electrodes, and that generates a plasma which is to be in contact with the liquid stored in the tank; a filter that is disposed so as to intersect the air flow and that causes the liquid stored in the tank and the gas in the case to come into contact with each other; and a controller that controls the fan and the power supply. The controller causes the power supply to start application of a voltage, and after elapse of a predetermined time period since the start of the application of the voltage, the controller causes the fan to start to drive.

With this configuration, the air purifier generates the plasma, which can increase the solubility of the substances to be decomposed in the liquid, before taking the substances into the case by the air flow. Thus, the substance can be effectively dissolved into liquid so as to easily come into contact with the active species produced by the plasma. As a result, the efficiency of decomposition of the substances can be increased.

For example, the controller may stop application of a voltage by the power supply in at least part of the time period in which the air flow is generated.

In this case, in at least part of the time period in which the air flow is generated, generation of a plasma is stopped, thereby avoiding production of a reactive gas such as an ozone gas or NOₓ. Therefore, it is possible to avoid leakage of a reactive gas with an air flow to the outside of the case.

For example, the controller may stop application of a voltage by the power supply concurrent with start of driving of the fan.

In this case, generation of a plasma is stopped at the same time when the fan starts to be driven, thereby avoiding production of a reactive gas such as an ozone gas or NOₓ. Therefore, it is possible to avoid leakage of a reactive gas with an air flow to the outside of the case.

For example, the air purifier may further include an input unit that receives input from a user. The controller may start to apply the voltage when the input unit receives the input.

Thus, it is possible for a user to generate a plasma at a desired timing, and to create a state in which substances to be decomposed are likely to be dissolved. For example, the input from a user may be to turn on the device.

An air purifier according to an aspect of the present disclosure includes: a case having an air inlet and an air outlet; a fan that generates an air flow so that a gas is taken into the case through the air inlet or a gas is discharged to the outside of the case through the air outlet; a tank that is disposed in the case and that stores a liquid; a plasma generator that includes a pair of electrodes and a power supply which applies a voltage between the pair of electrodes, and that generates a plasma which is to be in contact with the liquid stored in the tank; a filter that is disposed so as to intersect the air flow and that causes the liquid stored in the tank and the gas in the case to come into contact with each other; and a controller that selectively controls the fan and the power supply.

Thus, it is possible to reduce leakage of a reactive gas such as an ozone gas or NOₓ to the outside of the purifier.

For example, the controller, when generating a plasma, may start to apply a voltage by the power supply when the fan is not in operation or at the same time when the fan is stopped. The controller, when generating the air flow, may start to drive the fan when application of a voltage by the power supply is not in operation or at the same time when application of a voltage by the power supply is stopped.

Hereinafter, embodiments will be specifically described with reference to the drawings.

It is to be noted that each of the embodiments described below provides a comprehensive or specific example. The numerical values, shapes, materials, components, arrangement of the components, connection relationship between the components, steps, and order of the steps that are presented in the following embodiments are examples, which are not intended to limit the present disclosure. In the following embodiments, the components thereof, which are not described in the independent claim that defines the most generic concept of the present disclosure, are regarded as optional components. Each of the drawings is schematically illustrated, and is not necessarily illustrated accurately. In the drawings, the same constituent members are labeled with the same symbol.

### First Embodiment

### [1. Configuration of Air Purifier]

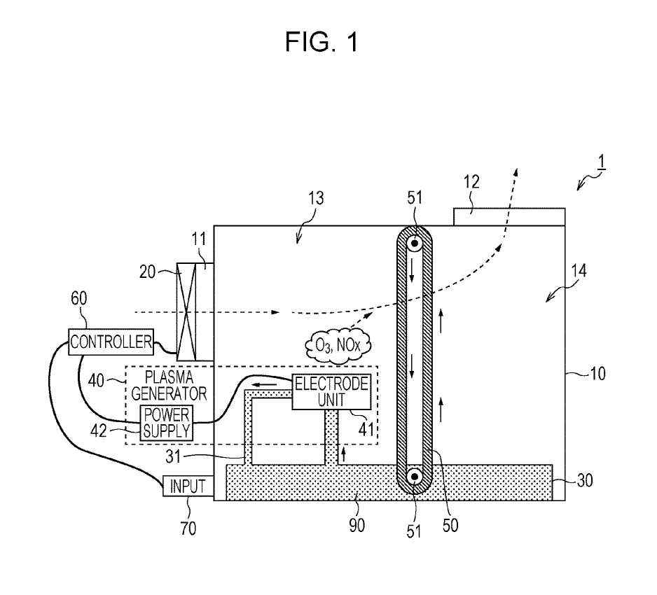

A configuration of an air purifier according to a first embodiment is given with reference to FIG. 1. FIG. 1 illustrates a configuration example of an air purifier 1 according to the first embodiment.

The air purifier 1 decomposes substances to be decomposed contained in air using a plasma. Specifically, the air purifier 1 generates a plasma in a liquid 90 to produce active species in the liquid 90, and then takes the substances into the liquid 90 so that the active species react with the substances, thereby decomposing the substances. The air purifier 1 may generate a plasma in the vicinity of the liquid 90. In this case, a gas produced by the plasma passes through the liquid 90, thereby supplying active species to the liquid 90.

The substances to be decomposed are specific substances contained in the air. The substances to be decomposed include, for example, toxic substances, contaminated substances, and odor substances. The toxic substances refer to, for example, chemical substances hazardous to humans and the ecosystem. The odor substances refer to, for example, chemical substances which are the source of bad smell (for example, odor of oil). The substances to be decomposed may be, for example, particulates or microorganisms. Examples of particulates include pollen and house dust.

The air purifier 1 is utilized as an air cleaner, for example. In this case, the air purifier 1 is disposed, for example, in predetermined space such as a room to purify the air in the space.

Alternatively, the air purifier 1 is utilized as a deodorization device or a disinfection device, for example. In this case, the air purifier 1 may be provided in a device that stores or cooks food, such as a refrigerator, a microwave oven, and a fryer. The air purifier 1 may be provided in an air conditioner, a humidifier, a washing machine, a dish washer, or a vehicle, for example.

### [2. Various Components of Air Purifier]

Various components of the air purifier 1 will be described in detail.

As illustrated in FIG. 1, the air purifier 1 includes a case 10, a fan 20, a tank 30, a plasma generator 40, a filter 50, a controller 60, and an input unit 70. The filter 50 is an example of a gas-liquid contact member.

### [2-1. Case]

The case 10 forms the contour of the air purifier 1. For example, the case 10 is composed of a resin material such as plastic and/or a metal material. The case 10 may have any shape. As illustrated in FIG. 1, the case 10 has an air inlet 11 and an air outlet 12.

The air inlet 11 is an opening for taking in air inwardly from the outside. The air outlet 12 is an opening for discharging the air in the case 10 outwardly. Air is taken into the case 10 through the air inlet 11 and passes through the filter 50, then is discharged to the outside of the case 10 through the air outlet 12 (see the dashed line arrow in FIG. 1).

In the example illustrated in FIG. 1, the air inlet 11 is provided in a lateral face of the case 10, and the air outlet 12 is provided in the upper face of the case 10. In this case, a long distance between the air inlet 11 and the air outlet 12 can ensure a space for providing the filter 50 and allow the air to smoothly flow from the air inlet 11 to the air outlet 12.

It is to be noted that the position where the air inlet 11 and the air outlet 12 are provided is not limited to the position in the above example. For example, the air inlet 11 and the air outlet 12 may be provided in any one of the upper face, a lateral face, and the lower face of the case 10. Specifically, the air inlet 11 may be provided in the upper face of the case 10, and the air outlet 12 may be provided in a lateral face of the case 10. Alternatively, both the air inlet 11 and the air outlet 12 may be provided in one face selected from a lateral face, the upper face, and the lower face. In the example illustrated in FIG. 1, the case 10 has one air inlet 11 and one air outlet 12. The case 10, however, may have multiple air inlets 11 and/or multiple air outlets 12. The air outlet 12 may be provided with a filter or the like that absorbs water content in the air.

The internal space of the case 10 is divided into a first space 13 and a second space 14 by the filter 50. The first space 13 is connected to the air inlet 11, and the second space 14 is connected to the air outlet 12.

### [2-2. Fan]

The fan 20 is an example of a blower that generates an air flow which flows to the air outlet 12 through the air inlet 11. In other words, the fan 20 generates an air flow from the first space 13 to the second space 14.

The fan 20 is provided in at least one of the air inlet 11 and the air outlet 12. When the fan 20 is provided in the air inlet 11, the fan 20 takes gas into the case 10 through the air inlet 11. Thus, a pressure difference occurs between the inside and outside of the case 10, and gas is discharged to the outside of the case 10 through the air outlet 12, for example. When the fan 20 is provided in the air outlet 12, the fan 20 discharges gas to the outside of the case 10 through the air outlet 12. Thus, a pressure difference occurs between the inside and outside of the case 10, and gas is taken into the case 10 through the air inlet 11, for example. Each of these is an example of the “fan that generates an air flow from the air inlet to the air outlet” in the present disclosure.

In the example illustrated in FIG. 1, the fan 20 is provided in the air inlet 11. The fan 20, however, may be provided only in the air outlet 12, or provided in both the air inlet 11 and the air outlet 12. For example, when the fan 20 is provided in both the air inlet 11 and the air outlet 12, these two fans 20 allow the air to flow from the air inlet 11 to the air outlet 12 more smoothly. It is to be noted that the fan 20 may be provided in the inside of the case 10.

The driving and stopping of the fan 20 are controlled by the controller 60. The details will be described later.

### [2-3. Tank]

The tank 30 is disposed in the case 10. The tank 30 stores the liquid 90. For example, the tank 30 has a box-shaped body with an opening in the upper face like a tray, and is disposed on the bottom of the case 10.

At first, the liquid 90 is pure water, for example. After generation of plasma, the liquid 90 contains active species. Examples of active species include hydroxyl radical (OH), hydrogen radical (H), oxygen radical (O), superoxide anion (O₂—), monovalent oxygen ion (O—), and hydrogen peroxide (H₂O₂). In addition, when a reactive gas containing a nitrogen monoxide (NO) gas and/or a nitrogen dioxide (NO₂) gas comes into contact with the liquid 90 to dissolve thereinto, the liquid 90 may contain nitrous acid (HNO₂) as an active species, for example.

It is to be noted that the liquid 90 may not be pure water and may be water containing predetermined compounds. For example, the liquid 90 may contain compounds for promoting decomposition of substances to be decomposed.

As illustrated in FIG. 1, a pipe 31 connected to the tank 30 may be provided. The pipe 31 is provided with part of the plasma generator 40. For example, the plasma generator 40 generates the plasma to produce active species in the liquid 90 in the pipe 31 so that the active species spread over the tank 30 via the pipe 31.

The pipe 31 is formed of a tubular member such as a pipe, a tube, or a hose, for example. In the example illustrated in FIG. 1, the pipe 31 is provided above the tank 30. For example, a pump (not illustrated) may be provided in the path of the pipe 31, and the pump circulates the liquid 90 in a predetermined direction (see the solid line arrow in FIG. 1).

The tank 30 and the pipe 31 are formed of, for example, a resin material or a metal material. It is to be noted that when the tank 30 and the pipe 31 are formed of a metal material, plating or coating processing may be performed on the surfaces thereof to protect against rust.

The air purifier according to the present embodiment may include no pipe 31. For example, the plasma generator 40 may generate a plasma so as to be in contact with the liquid 90 in the tank 30. In this case, for example, a pair of electrodes of the plasma generator 40 may be both disposed in the liquid 90. Alternatively, one or both of the electrodes may be disposed in the atmosphere so as not to be in contact with the liquid 90. In this case, for example, the plasma generator 40 generates a plasma in the atmosphere to produce active species, and then a gas containing the active species comes into contact with the liquid 90. In this manner, the air purifier may be formed in a simple configuration.

### [2-4. Plasma Generator]

The plasma generator 40 generates a plasma so as to be in contact with the liquid 90 stored in the tank 30, for example. The generated plasma produces active species in the liquid 90. For example, the plasma generator 40 is provided in the path of the pipe 31, and generates a plasma in the liquid 90 in the pipe 31.

For example, the plasma generator 40 includes an electrode unit 41 and a power supply 42. The electrode unit 41 includes a pair of electrodes. These electrodes are at least partly disposed in the pipe 31 and are spaced from each other. The power supply 42 applies a voltage between the pair of electrodes. For example, the power supply 42 applies a negative-polarity high voltage pulse having 2 to 50 kV/cm, 100 Hz to 100 kHz between the pair of electrodes to cause electric discharge in the liquid 90.

A bubble is generated near the pair of electrodes in the liquid 90 in the pipe 31 by evaporation of the liquid 90 due to the energy of the electric discharge, and vaporization of the liquid 90 due to a shock wave generated by the electric discharge. The plasma generator 40 generates a plasma in the bubble to produce active species in the liquid 90. Accordingly, the active species exist in abundance near the electrode unit 41, thus efficiently decomposing a substance therenear.

In the example illustrated in FIG. 1, the plasma generator 40 is provided in the first space 13 between the air inlet 11 and the filter 50. When the plasma generator 40 generates the plasma, a highly reactive gas such as ozone gas (O₃) or NO (e.g., NO, NO₂) is produced. For example, the reactive gas produced in the first space 13 is dissolved in the liquid 90 in the filter 50.

The driving and stopping of the plasma generator 40 are controlled by the controller 60. Specifically, starting and stopping of application of a voltage using the power supply 42 are controlled by the controller 60.

The air purifier 1 may further include a pump (not illustrated) that supplies a gas in the vicinity of at least one of the electrodes of the pair. In this case, the pump may generate a bubble near the pair of electrodes, and the plasma generator 40 may generate a plasma in the bubble. The pump supplies, for example, the gas in the first space 13 or the air outside the case 10 to the vicinity of at least one of the electrodes of the pair.

The pump can eliminate the need for vaporizing the liquid 90 with electric discharge, thereby enabling the power of the plasma generator 40 to be effectively utilized for the generation of a plasma. Consequently, it is possible to reduce power consumption.

### [2-5. Filter]

The filter 50 is provided between the air inlet 11 and the air outlet 12. The filter 50 is disposed so as to intersect the air flow generated by the fan 20. The filter 50 is disposed so as to divide the space in the case 10 into the first space 13 and the second space 14. The liquid 90 is supplied from the tank 30 to the filter 50, and the air taken into the case 10 pass through the filter 50. Thus, the liquid 90 comes into contact with the air via the filter 50.

The filter 50 is provided so that no gap occurs between the filter 50 and each lateral face of the case 10 and between the filter 50 and the upper face of the case 10, for example. Thus, most of the air taken in through the air inlet 11 is allowed to pass through the filter 50.

The filter 50 may be a member that increases the area of the liquid 90 in contact with air. For example, the filter 50 may be a porous member composed of stainless steel or chemical materials. The porous member is, for example, a porous plate having fine pores. It is to be noted that the average pore diameter of the filter 50 is, for example, several mm or less. The average pore diameter of the filter 50 is obtained by averaging the diameters of the pores found in any section of the filter 50, for example. It is to be noted that when the shape of a pore is not circular, the diameter of the pore is equivalent to the diameter of a circle which has the same area as the area of the pore. These pores catch air, and thus the air and the liquid 90 are likely to come into contact with each other. It is to be noted that the filter 50 is able to catch not only the air taken into the case 10, but also the gas produced by the plasma generator 40, for example.

Alternatively, the filter 50 may be a cloth-like member that has breathability and water absorbability. The filter 50 may have raised fibers in order to increase the surface area.

In the example illustrated in FIG. 1, the filter 50 has a wide loop belt-like shape, and is stretched between a pair of pulleys 51. The filter 50 is rotated by the pair of pulleys 51.

In the example illustrated in FIG. 1, the filter 50 is rotated by the pulleys 51 while part of the filter 50 is immersed in the liquid 90 of the tank 30 (see the solid line arrow in FIG. 1). Thus, the filter 50 entirely soaking with the liquid 90 is exposed to the air. Thus, substances to be decomposed in the air are allowed to come into contact with the active species in the liquid 90 via the filter 50, and can be decomposed by a reaction with the active species.

Part of the substances, which has been taken into the liquid 90 in the filter 50 but not been decomposed, may be carried into the tank 30 with the rotation of the pulleys 51 to be decomposed in the tank 30 or in the pipe 31. The liquid 90 in the tank 30 and the pipe 31 may contain more active species than the liquid 90 in the filter 50.

### [2-6. Controller]

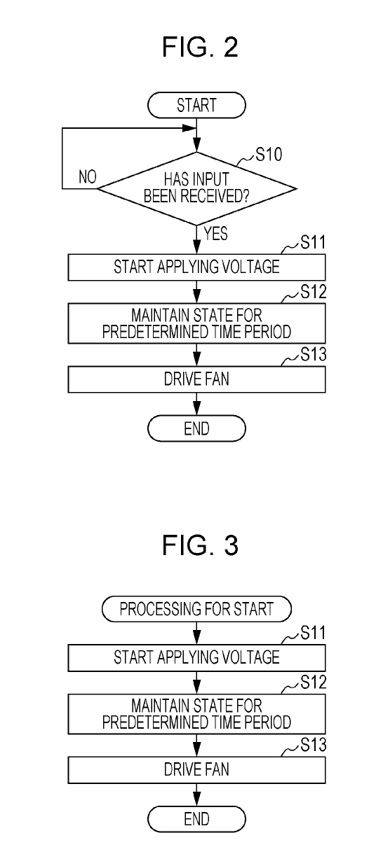

The controller 60 controls the fan 20 and the power supply 42. The controller 60 causes the power supply 42 to start application of a voltage, and after elapse of a predetermined time period since the start of the application of a voltage, the controller 60 starts to drive the fan 20. In other words, the controller 60 controls the plasma generator 40 to generate a plasma, and subsequently, controls the fan 20 to generate an air flow. For example, the controller 60 may control the power supply 42 to start application of a voltage in response to input from the input unit 70. A specific example of the control by the controller 60 will be described later with reference to FIGS. 2 to 6B.

The controller 60 has, for example, a nonvolatile memory that stores a program, a volatile memory which is a temporary storage area for executing a program, an input/output port, and a processor for executing a program. The controller 60 is, for example, a microcomputer.

### [2-7. Input Unit]

The input unit 70 is an input interface.

The input unit 70 is a user interface that receives input from a user, for example. For example, the input unit 70 receives a command to start purification treatment of air. The input unit 70 may further receive a command to stop purification treatment of air.

In this case, the input unit 70 may be a depression-type physical button, and is provided outside the case 10. When a user presses the button, the input unit 70 notifies the controller 60 of the input from a user. The input unit 70 may be a slide switch or a touch panel.

Alternatively, the input unit 70 may be a communication interface. In this case, the input unit 70 may receive a control command from a controller (not illustrated), for example. The controller may be a smartphone operated by a user, for example.

### [3. Operation]

The operation of the air purifier 1 according to the present embodiment will be described with reference to FIG. 2. FIG. 2 is a flow chart illustrating a first example of operation of the air purifier 1 according to the first embodiment. In other words, FIG. 2 is a flow chart illustrating an example of an air purification method according to the present embodiment.

The air purifier 1 starts the operation in response to energization of the power supply of the main body. At this point, the fan 20 is not in operation.

First, the controller 60 stands by until the input unit 70 receives input from a user (No in S10). The input from a user is, for example, a command to start purification treatment of air. When the input unit 70 receives input from a user (Yes in S10), the controller 60 causes the power supply 42 to start application of a voltage (S11). Thus, the power supply 42 applies a predetermined high-voltage pulse between a pair of electrodes, and generates a plasma in the liquid 90.

The controller 60 maintains a state in which the fan 20 is not in operation and the power supply 42 applies the voltage, for a predetermined time period from the start of the application of the voltage (S12).

The controller 60 may have a timer and a comparator, for example. In this case, for example, the timer measures elapsed time since the start of the application of a voltage, and the comparator may compare the measured elapsed time with a predetermined time period. When the elapsed time reaches a predetermined time period, the controller 60 starts control for subsequent steps (S13).

The controller 60 causes the fan 20 to start generation of an air flow, for example, after the solubility of odor substances in the liquid 90 reaches a predetermined level. For example, in the case where a time period taken for the solubility to reach a predetermined level has been obtained experimentally, the time period may be preset in the controller 60 to determine whether or not the solubility has reached a predetermined level. The predetermined level is, for example, 1.5 times the solubility before the start of the application of a voltage.

Subsequently, the controller 60 starts to drive the fan 20 (S13). Specifically, the fan 20 starts to rotate, and thereby air is started to be taken into the case 10 through the air inlet 11.

The air taken in forms an air flow in the case 10 and then comes into contact with the liquid 90 via the filter 50. Furthermore, the controller 60 starts to rotate the filter 50 with the pulleys S1 to promote contact between the air and the liquid 90, for example. The air, after coming into contact with the liquid 90, is discharged through the air outlet 12.

It is to be noted that when the air purifier 1 further includes a fan in the air outlet 12, the controller 60 may start to drive the fan 20 of the air inlet 11 and the fan of the air outlet 12 simultaneously.

In the example illustrated in FIG. 2, a predetermined time period is provided from the start of application of a voltage until the fan 20 starts to be driven. Therefore, it is possible to increase the solubility of substances to be decomposed in the liquid 90 by the plasma before the fan 20 starts to be driven. Thus, after the fan 20 starts to be driven, the substances taken in with air are likely to be dissolved in the liquid 90, and therefore are likely to come into contact with the active species in the liquid 90. Consequently, the substances can be efficiently decomposed.

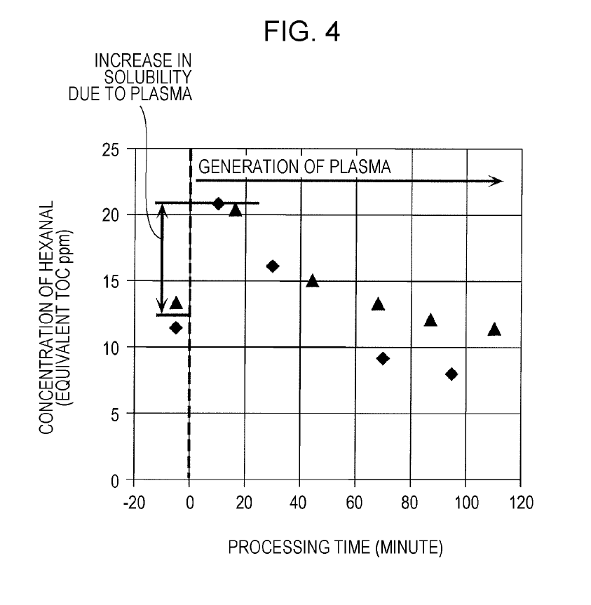

In the example illustrated in FIG. 2, the purification treatment of air is started based on input from a user. The present embodiment, however, is not limited to this. FIG. 3 is a flow chart illustrating a second example of operation of the air purifier 1 according to the present embodiment.

As illustrated in FIG. 3, the controller 60 may start application of a voltage without receiving input from a user (S11). For example, the controller 60 may cause the power supply 42 to start application of a voltage in response to energization of the air purifier 1. The processing after this step is the same as the processing S11, S12, and S13 described with reference to FIG. 2. In this case, the air purifier 1 may not include the input unit 70.

### [4. Experimental Result 1]

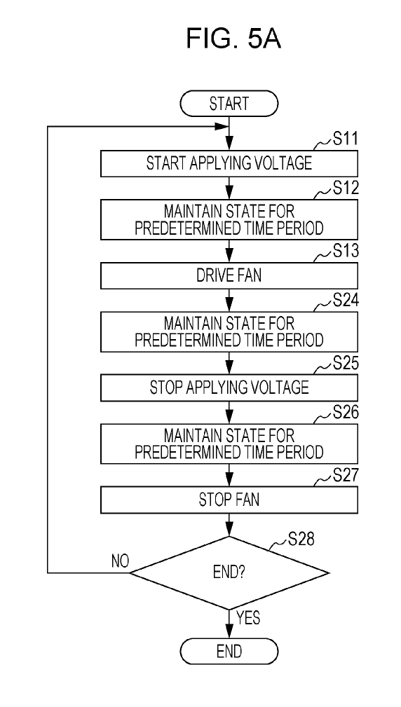

An experimental result related to the relationship between generation of a plasma and the solubility of substances to be decomposed in the liquid 90 will be described with reference to FIG. 4. FIG. 4 is a graph illustrating the relationship between generation of a plasma and the solubility of odor substances.

In the experiment, the air purifier 1 is prepared which is in a state where odor substances are floating on the liquid surface of the tank 30. In this state the plasma generator 40 was operated, and total organic carbon (TOC) concentration in the liquid 90 was measured. FIG. 4 illustrates the measurement result.

In FIG. 4, the horizontal axis indicates the time of plasma treatment. Here, the time at which application of a voltage is started is 0. The vertical axis indicates a TOC concentration to which the concentration of hexanal in the liquid 90 is converted. An experiment was conducted twice and the result of each experiment was illustrated by the rhombus plots and the triangle plots in FIG. 4.

Hexanal (C₆H₁₂O), a type of chain aliphatic aldehyde, is an example of odor substances which become a source of bad smell. For example, hexanal is an oil odor chemical substance which is produced when aliphatic acid contained in oils and fats is oxidized.

Before a plasma was generated, the TOC concentration was approximately 12 ppm. From this, it may be seen that part of the hexanal dropped on the liquid surface was dissolved in the liquid 90.

The TOC concentration was 20 ppm when 10 minutes had elapsed since the generation of a plasma. That is, the TOC concentration was higher than before the generation of a plasma. From this, it may be seen that a large amount of hexanal was dissolved in the liquid 90 due to the generation of a plasma.

Subsequently 120 minutes later, the TOC concentration decreased to approximately 10 ppm. Similar results were obtained in each of the two experiments. From this, it may be seen that active species produced by the plasma decomposed the hexanal.

The experimental results demonstrate that the solubility of odor substances such as hexanal may be significantly increased due to the generation of a plasma.

It is presumed that the solubility reaches a desired level after elapse of a time period since the start of generation of a plasma. Therefore, the time period may be provided before the odor substances are taken into the case 10 by air flow. This enables the odor substance to be efficiently dissolved into the liquid 90.

### [5. Other Operations]

Another example of the operation of the air purifier 1 according to the present embodiment will be described.

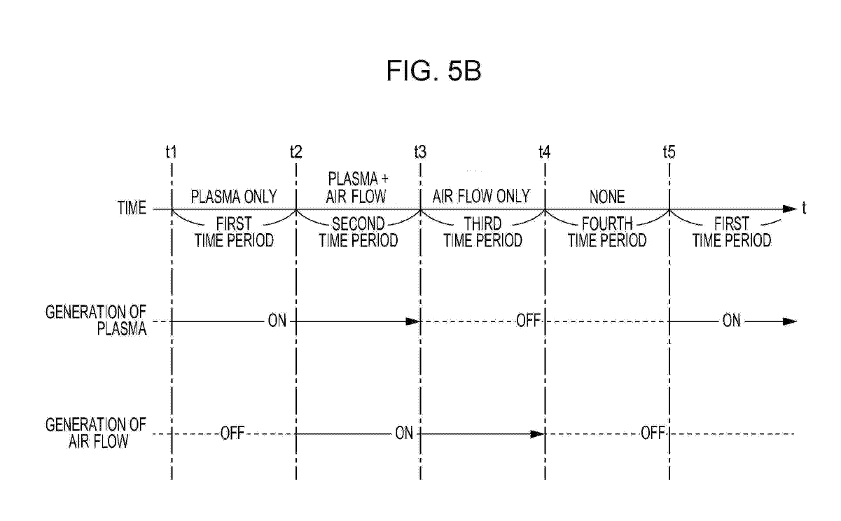

FIG. 5A is a flow chart illustrating a third example of the operation of the air purifier 1. FIG. 5B is a timing chart illustrating the timing of generation of a plasma and the timing of generation of an air flow in the operation illustrated in FIG. 5A.

Here, a state where the power supply 42 applies a voltage between a pair of electrodes and the fan 20 generates no air flow may be called a first state. A state where the power supply 42 applies a voltage between a pair of electrodes and the fan 20 generates an air flow may be called a second state. A state where the power supply 42 applies no voltage between a pair of electrodes and the fan 20 generates an air flow may be called a third state. A state where the power supply 42 applies no voltage between a pair of electrodes and the fan 20 generates no air flow may be called a fourth state. A time period in which the first state is maintained is called a first time period, a time period in which the second state is maintained is called a second time period, a time period in which the third state is maintained is called a third time period, and a time period in which the fourth state is maintained is called a fourth time period.

The processing S11 to S13 in FIG. 5A is the same as the processing S11 to S13 in FIG. 3.

The controller 60 starts to apply a voltage at time t1 (S11). Thus, a plasma is generated and thereby active species are supplied to the liquid 90. The controller 60 maintains the first state for a predetermined time period (S12).

In the first period, only the generation of a plasma is continued. Thus, the solubility of substances to be decomposed in the liquid 90 is improved.

The controller 60 starts to drive the fan 20 at time t2 (S13). Subsequently, the controller 60 maintains the second state for a predetermined time period (S24).

In the second time period, generation of a plasma and generation of an air flow are continued. Thus, air is taken into the case 10 from the outside of the air purifier 1 by the fan 20, and then the taken-in air comes into contact with the liquid 90 via the filter 50. Thus, substances contained in the air are decomposed by active species in the liquid 90, and then purified air is discharged through the air outlet 12.

The second time period may be preset to the controller 60, for example. Alternatively, the end point of the second time period may be determined, for example, by input of a command from a user to stop purification treatment of air.

The controller 60 causes the power supply 42 to stop application of a voltage at time t3 (S25). Thus, generation of a plasma is stopped, and thereby supply of the active species to the liquid 90 is stopped. The controller 60 maintains the third state for a predetermined time period (S26).

In the third time period, only the generation of an air flow is continued. Air is taken into the case 10, and then comes into contact with the liquid 90 via the filter 50. Thus, substances contained in the air are decomposed by active species remaining in the liquid 90, and then purified air is discharged through the air outlet 12. On the other hand, since the application of a voltage has been stopped, electric discharge does not occur and thus a reactive gas such as an ozone gas or NO is not produced. Therefore, the amount of reactive gas contained in the gas flowing out through the air outlet 12 is reduced.

The controller 60 stops the fan 20 at time t4 (S27). Thus, generation of an air flow in the case 10 is stopped.

When the purification treatment of air is terminated (Yes in S28), the processing is terminated. When the purification treatment of air is not terminated (No in S28), the flow returns to step S11.

When the purification treatment is not terminated, as illustrated in FIG. 5B, the controller 60 may maintain the fourth state for a predetermined time period after stopping the fan 20, and subsequently may start application of a voltage.

Alternatively, the controller 60 may start application of a voltage at the same time of stopping the fan 20. In other words, the fourth time period may not be provided.

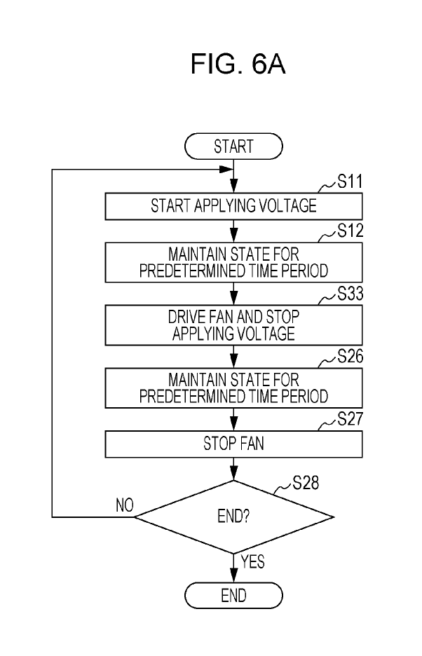

FIG. 6A is a flow chart illustrating a fourth example of the operation of the air purifier 1. FIG. 6B is a timing chart illustrating the timing of generation of a plasma and the timing of generation of an air flow based on the operation illustrated in FIG. 6A.

The air purification method illustrated in FIG. 6A has the processing S33 instead of the processing S13, S24, S25 illustrated in FIG. 5A.

The controller 60 starts to drive the fan 20 at time t2 (=t3), and simultaneously causes the power supply 42 to stop application of a voltage (S33). In other words, the controller 60 selectively drives one of the power supply 42 and the fan 20 so that a state (i.e., the second state) where a plasma is generated and an air flow is generated. Thus, it is possible to reduce leakage of a reactive gas produced by the plasma to the outside of the case 10 by an air flow.

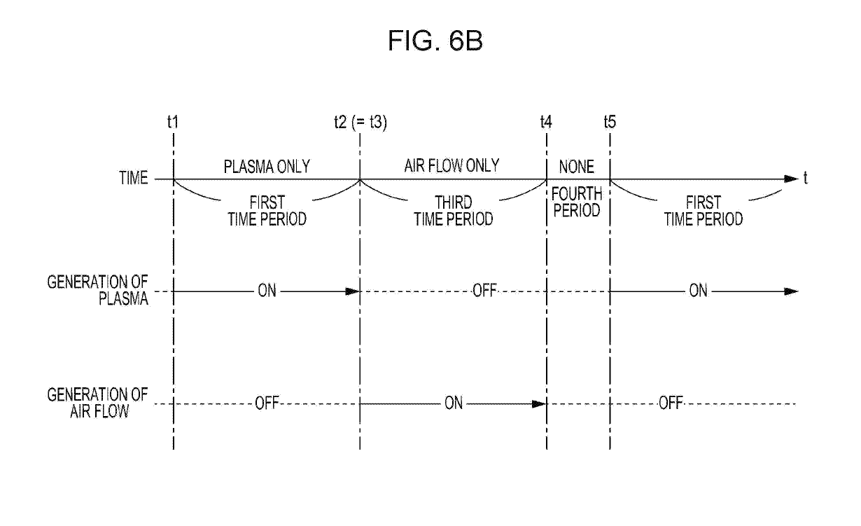

FIG. 7 is a flow chart illustrating a fifth example of the operation of the air purifier 1.

As illustrated in FIG. 7, the controller 60 causes the power supply 42 to stop application of a voltage, and simultaneously drives the fan 20 (S41). In other words, the controller 60 generates an air flow without generating a plasma. The controller 60 maintains the third state for a predetermined time period (S42).

It is to be noted that when the power supply 42 is not applying a voltage at the start time illustrated in FIG. 7, the processing S41 may only drive the fan 20.

Subsequently, the controller 60 stops driving of the fan 20 and simultaneously causes the power supply 42 to start application of a voltage (S43). In other words, the controller 60 generates a plasma without generating an air flow. The controller 60 maintains the first state for a predetermined time period (S44).

When the purification treatment of air is terminated (Yes in S45), the processing is terminated. When the purification treatment of air is not terminated (No in S45), the flow returns to step S41.

In the example illustrated in FIG. 7, drive of the fan 20 and application of a voltage by the power supply 42 are performed alternately. Thus, it is possible to reduce leakage of a reactive gas such as an ozone gas or NO to the outside of the case 10 by an air flow.

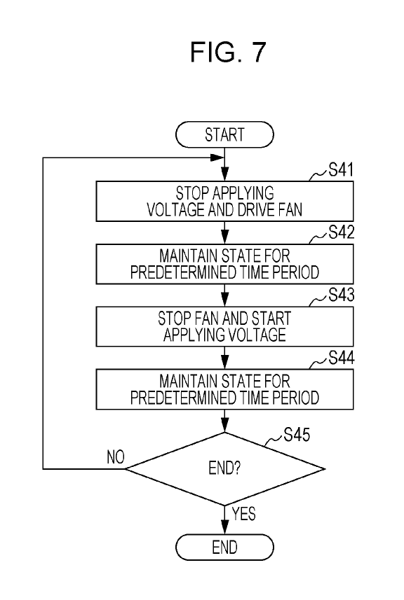

FIG. 8 is a flow chart illustrating a sixth example of the operation of the air purifier 1.

The controller 60 causes the power supply 42 to start application of a voltage (S51). The controller 60 maintains the first state for a predetermined time period (S52).

Subsequently, the controller 60 causes the power supply 42 to stop application of a voltage (S53). The controller 60 maintains the fourth state for a predetermined time period (S54).

Subsequently, the controller 60 causes the fan 20 to drive (S55). The controller 60 maintains the third state for a predetermined time period (S56).

Subsequently, the controller 60 stops the fan 20 (S57). The controller 60 maintains the fourth state for a predetermined time period (S58).

When the purification treatment of air is terminated (Yes in S59), the processing is terminated. When the purification treatment of air is not terminated (No in S59), the flow returns to step S51.

In the example illustrated in FIG. 8, after elapse of a predetermined time since the stop of the fan 20, the controller 60 causes the power supply 42 to start application of a voltage to generate a plasma. Similarly, after elapse of a predetermined time since the stop of the application of a voltage, the controller 60 causes the fan 20 to drive. In other words, the controller 60 causes generation of a plasma and generation of an air flow alternately with a standby time period provided therebetween.

Thus, it is possible to further reduce leakage of a reactive gas such as an ozone gas or NO to the outside of the air purifier 1. For example, in the standby time period after the stop of application of a voltage, a reactive gas produced by inertia may be dissolved in the liquid 90. Similarly, in the standby time period after the stop of the fan 20, an air flow possibly generated by inertia ceases, and thus it is possible to avoid leakage of a reactive gas with an air flow to the outside of the case 10.

### [6. Experimental Result 2]

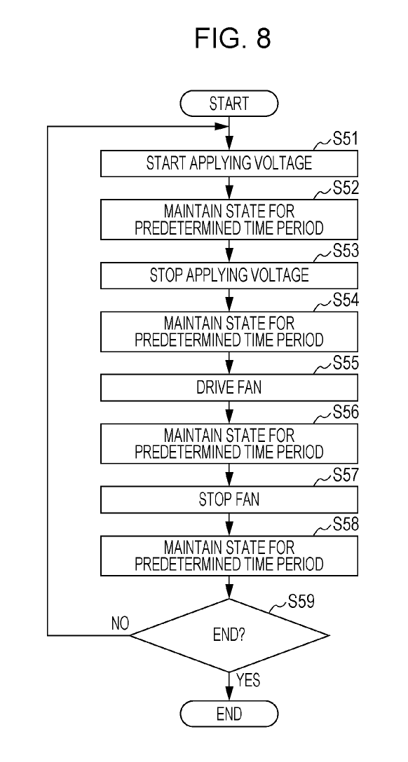

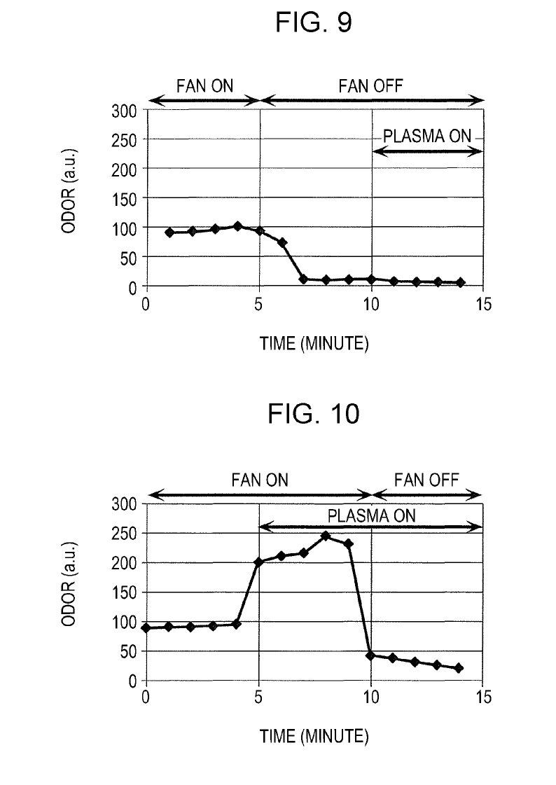

In the following, an experimental result of the concentration of odor substances, measured by using a volatile organic compound (VOC) sensor, in the vicinity of the air outlet 12 of the air purifier 1 according to the first embodiment will be described with reference to FIGS. 9 and 10.

FIG. 9 illustrates a variation in the concentration of odor substances in the vicinity of the air outlet 12 when a standby time period is provided between generation of a plasma and generation of an air flow. FIG. 10 illustrates a variation in the concentration of odor substances in the vicinity of the air outlet 12 when a time period is provided in which a plasma and an air flow are generated concurrently.

The odor substances detected by a VOC sensor include not only the odor substances (e.g., oil odor) contained in the air, but also a reactive gas such as an ozone gas or NO produced by a plasma.

As illustrated in FIG. 9, in a time period for 0 to 5 minutes, the fan 20 was in operation and thereby an air flow was generated. Thus, odor substances contained in the air were detected. In the time period of 5 to 10 minutes, the fan 20 was stopped and thereby no gas flowed out through the air outlet 12. Thus, almost no odor substances were detected. In a time period of 10 to 15 minutes, a plasma was generated in a state where the fan 20 was stopped. Since the fan 20 was stopped, almost no odor substances were detected.

On the other hand, as illustrated in FIG. 10, in a time period of 5 to 10 minutes, a plasma and an air flow were generated concurrently. Thus, odor substances were detected and the concentration of the odor substances was higher than the concentration of the odor substances contained in the air.

### Second Embodiment

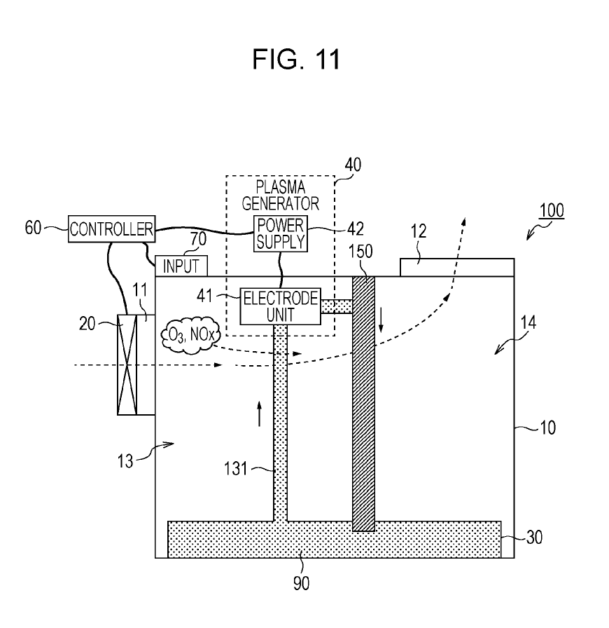

An air purifier according to a second embodiment will be described with reference to FIG. 11. FIG. 11 illustrates an example of a configuration of an air purifier 100 according to the second embodiment.

The air purifier 100 illustrated in FIG. 11 differs from the air purifier 1 illustrated in FIG. 1 in that the air purifier 100 includes a pipe 131 and a filter 150 instead of the pipe 31 and the filter 50. In the following, points of difference will be mainly described.

The pipe 131 supplies the liquid 90 stored in the tank 30 to the filter 150. For example, the pipe 131 is formed of a tubular member such as a pipe, a tube, or a hose. For example, a pump (not illustrated) may be provided in the path of the pipe 131. In this case, the liquid 90 may be sucked up by the pump from the tank 30 and be supplied to an upper portion of the filter 150.

The plasma generator 40 is provided in the pipe 131. The plasma generator 40 supplies active species to the liquid 90 that flows through in the pipe 131. Thus, the liquid 90 containing the active species is supplied to the filter 150. Consequently, the efficiency of decomposition of the substances to be decomposed by the filter 150 can be increased.

The filter 150 is provided between the air inlet 11 and the air outlet 12. The liquid 90 stored in the tank 30 comes into contact with the air in the case 10 via the filter 150. The filter 150 is disposed so as to intersect the air flow generated by the fan 20. In the example illustrated in FIG. 11, the liquid 90 is supplied to an upper portion of the filter 150 by the pipe 131. In FIG. 11, the air purifier 100 does not include the pulleys 51, and the filter 150 does not rotate. For this reason, various types of members may be utilized as the filter 150. For example, the filter 150 may be an aggregate of beads (or granular materials)

In the example illustrated in FIG. 11, since the filter 150 stands still, intimate contact between the filter 150 and another member (e.g., the inner surface of the case 10) may be facilitated, and thus the space between them may be made small. Consequently, most of the gas taken into the case 10 is allowed to pass through the filter 150, and thus the efficiency of decomposition of the substances to be decomposed may be increased.

The liquid 90 supplied to an upper portion of the filter 150 flows into the tank 30 through the filter 150. The liquid 90 collected by the tank 30 is again supplied to the upper portion of the filter 150 by the pipe 131. That is, the air purifier 100 has a circulation path including the tank 30, the pipe 131, and the filter 150, and the liquid 90 containing active species circulates along the circulation path. Thus, substances to be decomposed may be efficiently taken in the liquid 90, and the taken-in substances can be efficiently decomposed.

It is to be noted that the air purifier 100 according to the second embodiment is capable of performing various operations described in the first embodiment.

### Other Embodiments

The air purifier and the air purification method according to various embodiments have been described by way of illustration in the above. The present disclosure, however, is not limited to these embodiments. The present disclosure includes those embodiments to which various modifications which will occur to those skilled in the art are made and in which components in different embodiments are combined as long as the modifications do not depart from the spirit of the present disclosure.

For example, in the embodiments described above, an example has been illustrated in which the liquid 90 is supplied to the filter 50 by rotating the filter 50, and another example has been illustrated in which the liquid 90 is supplied to the filter 150 via the pipe 131. However, a method of supplying a liquid to a filter is not limited to this. For example, the air purifier may include a capillary tube as an example of the gas-liquid contact member. The capillary tube may suck up the water in the tank 30 utilizing capillary phenomenon.

For example, disposition of the air inlet 11, the air outlet 12, the tank 30, and the filter 50 is not particularly limited. For example, the air inlet 11 may be located in the lower surface of the case 10, and the air outlet 12 may be located in the upper surface of the case 10. In this case, the filter 50 may be disposed horizontally so as to face the upper surface of the case 10.

For example, in the embodiments described above, an example has been illustrated in which the plasma generator 40 is disposed in the first space 13. The embodiments, however, are not limited to this. The plasma generator 40 may be disposed in the second space 14.

It is to be noted that the present disclosure may be implemented not only as an air purifier, but also as an air purification method or a program that includes steps of processing to be performed by the components of the air purifier, and as a recording medium such as a computer readable digital versatile disc (DVD) on which the program is recorded.

In other words, comprehensive or specific embodiments may be implemented as a system, a device, an integrated circuit, a computer program, or a computer readable recording medium, or any selective combination thereof.

Various modifications, replacements, additions, and omissions may be made to the embodiments described above within the scope of the claims and the scope of the equivalents thereof.

The air purifier of the present disclosure is applicable to, for example, a deodorization device, a disinfection device, and an air cleaner.

What is claimed is:

1. An air purifier comprising:
a case having an air inlet and an air outlet;
a tank that stores a liquid, the tank disposed in the case;
a plasma generator that generates a plasma which is to be in contact with the liquid, the plasma generator including a pair of electrodes and a power supply which applies a voltage between the pair of electrodes;
a fan that generates an air flow from the air inlet to the air outlet;
a filter through which the air flow comes into contact with the liquid, the filter disposed in the case; and
a controller that
causes the power supply to start application of the voltage, and
causes the fan to start generation of the air flow after elapse of a predetermined time period since the start of the application of the voltage.

2. The air purifier according to claim 1,
wherein the controller further
causes the power supply to stop the application of the voltage, and
causes the fan to stop the generation of the air flow after the stop of the application of the voltage.

3. The air purifier according to claim 2,
wherein the controller causes the power supply to stop the application of the voltage at the same time when the controller causes the fan to start the generation of the air flow.

4. The air purifier according to claim 1, further comprising an input interface,
wherein the controller causes the power supply to start the application of the voltage in response to input from the input interface.

5. The air purifier according to claim 1,
wherein the air flow contains an odor substance, and
the controller causes the fan to start the generation of the air flow after a solubility of the odor substance in the liquid reaches a predetermined level.
